(19) United States
(12) Patent Application Publication
(10) Pub. No.: US 2024/0366207 A1
(43) Pub. Date: Nov. 7, 2024

(54) PATIENT TRACKING DEVICE FOR USE WITH MEDICAL INSTRUMENT NAVIGATION SYSTEM

(71) Applicants: Acclarent, Inc., Irvine, CA (US); Biosense Webster (Israel) Ltd., Yokneam (IL)

(72) Inventors: George Gusein, Acre (IL); Christopher A. Schutt, Bozeman, MT (US); Cesar Fuentes-Ortega, Pasadena, CA (US); Henry F. Salazar, Pico Rivera, CA (US); Raymond Yue-sing Tang, Rosemead, CA (US)

(21) Appl. No.: 18/594,170

(22) Filed: Mar. 4, 2024

Related U.S. Application Data
(60) Provisional application No. 63/464,257, filed on May 5, 2023.

(51) Int. Cl. A61B 17/02; A61B 17/00

(52) U.S. Cl. CPC A61B 17/0218; A61B 2017/00022; A61B 2017/00566; A61B 2017/0212; A61B 2017/0225

(57) ABSTRACT

An apparatus includes a tray, a deformable material, and a position sensor. The tray is configured to correspond to at least a portion of an alveolar ridge of a patient. The deformable material is configured to receive dentition of the alveolar ridge of the patient and thereby secure the tray relative to the alveolar ridge of the patient. The position sensor is fixed relative to the tray and is configured to generate a signal indicating a real-time position of the position sensor within three-dimensional space. Another apparatus includes a body, a position sensor, and a suction conduit. The body defines a cavity and is configured to fit in a mouth of a patient. The suction conduit is in fluid communication with the cavity and is configured to apply suction to the cavity to thereby secure the body to a cheek wall in the mouth of the patient.

### PRIORITY

This application claims priority to U.S. Provisional Pat. App. No. 63/464,257, entitled “Patient Tracking Device for Use with Medical Instrument Navigation System,” filed May 5, 2023, the disclosure of which is incorporated by reference herein, in its entirety.

### BACKGROUND

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. An example of an electromagnetic IGS navigation system that may be used in IGS procedures is the CARTO® 3 System by Biosense-Webster, Inc., of Irvine, California. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, some instruments can include sensors (e.g., electromagnetic coils that emit electromagnetic fields and/or are responsive to externally generated electromagnetic fields), which can be used to perform the procedure while the sensors send data to the computer indicating the current position of each sensor-equipped instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

In some instances, it may be desirable to track the real-time position of a patient relative to a fixed frame of reference (e.g., an electromagnetic field) while simultaneously tracking the real-time position of one or more medical instruments relative to the same fixed frame of reference. This may enable an IGS system to account for any movement of the patient while tracking the real-time position of one or more medical instruments, to thereby enable the IGS system to inform the operator of the real-time position of one or more medical instruments relative to anatomical structures of the patient. Some patient tracking devices may be secured to the skin of the patient. However, due to the elasticity of the skin of the patient, it is possible that a skin-mounted patient tracking device might not always provide accurate and reliable position data on certain anatomical structures of the patient. While several systems and methods have been made and used in connection with IGS navigation systems, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 depicts a schematic view of an example of a surgery navigation system being used on a patient seated in an example of a medical procedure chair;

FIG. 2 depicts a perspective view of an example of a patient tracking assembly, with a deformable material of the patient tracking assembly in a first state;

FIG. 3 depicts a perspective view of the patient tracking assembly of FIG. 2, with the deformable material in a second state;

FIG. 4 depicts a side elevational view of the patient tracking assembly of FIG. 2;

FIG. 5 depicts a schematic view of the patient tracking assembly of FIG. 2 installed in a mouth of a patient;

FIG. 6 depicts a perspective view of another example of a patient tracking assembly;

FIG. 7 depicts a perspective view of another example of a patient tracking assembly; and

FIG. 8 depicts a schematic view of the patient tracking assembly of FIG. 7 installed in a mouth of a patient.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. EXAMPLE OF AN IMAGE GUIDED SURGERY NAVIGATION SYSTEM

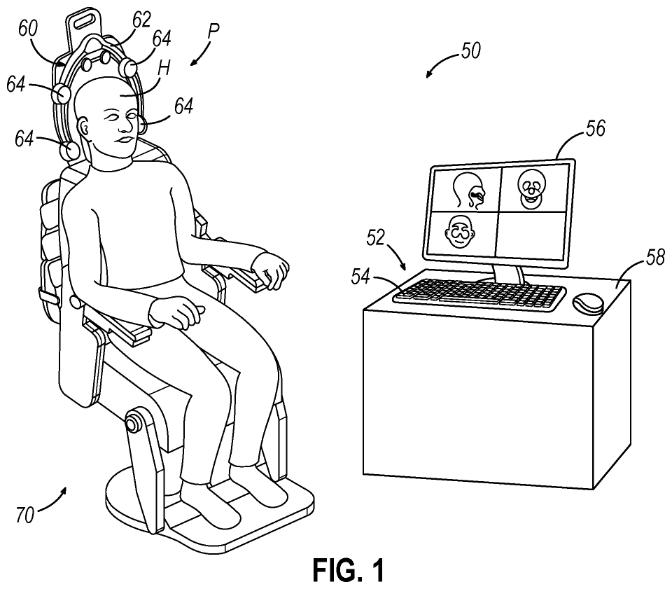

When performing a medical procedure within a head of a patient (P), it may be desirable to have information regarding the position of an instrument within the head (H) of the patient (P), particularly when the instrument is in a location where it is difficult or impossible to obtain an endoscopic view of a working element of the instrument within the head of the patient (P). FIG. 1 shows an example of an IGS navigation system (50) enabling a medical procedure to be performed within a head (H) of a patient (P) using image guidance. In addition to or in lieu of having the components and operability described herein IGS navigation system (50) may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein, in its entirety; and/or U.S. Pat. No. 10,561,370, entitled “Apparatus to Secure Field Generating Device to Chair,” issued Feb. 18, 2020, the disclosure of which is incorporated by reference herein, in its entirety.

IGS navigation system (50) of the present example comprises a field generator assembly (60), which comprises a set of magnetic field generators (64) that are integrated into a horseshoe-shaped frame (62). Field generators (64) are operable to generate alternating magnetic fields of different frequencies around the head (H) of the patient (P). An instrument may be inserted into the head (H) of the patient (P). Such an instrument may include one or more position sensors as described in greater detail below. In the present example, frame (62) is mounted to a chair (70), with the patient (P) being seated in the chair (70) such that frame (62) is located adjacent to the head (H) of the patient (P). By way of example only, chair (70) and/or field generator assembly (60) may be configured and operable in accordance with at least some of the teachings of U.S. Pat. No. 10,561,370, entitled “Apparatus to Secure Field Generating Device to Chair,” Issued Feb. 18, 2020, the disclosure of which is incorporated by reference herein, in its entirety. In some other variations, the patient (P) lies on a table; and field generator assembly (60) is positioned on or near the table.

IGS navigation system (50) of the present example further comprises a processor (52), which controls field generators (64) and other elements of IGS navigation system (50). For instance, processor (52) is operable to drive field generators (64) to generate alternating electromagnetic fields; and process signals from the instrument to determine the location of a navigation sensor or position sensor in the instrument within the head (H) of the patient (P). Processor (52) comprises a processing unit (e.g., a set of electronic circuits arranged to evaluate and execute software instructions using combinational logic circuitry or other similar circuitry) communicating with one or more memories. Processor (52) of the present example is mounted in a console (58), which comprises operating controls (54) that include a keypad and/or a pointing device such as a mouse or trackball. A physician uses operating controls (54) to interact with processor (52) while performing the surgical procedure.

While not shown, the instrument that is used with IGS navigation system (50) may include a navigation sensor or position sensor that is responsive to positioning within the alternating magnetic fields generated by field generators (64). A coupling unit (not shown) may be secured to the proximal end of the instrument and may be configured to provide communication of data and other signals between console (58) and the instrument. The coupling unit may provide wired or wireless communication of data and other signals.

In some versions, the navigation sensor or position sensor of the instrument may comprise at least one coil at or near the distal end of the instrument. When such a coil is positioned within an alternating electromagnetic field generated by field generators (64), the alternating magnetic field may generate electrical current in the coil, and this electrical current may be communicated along the electrical conduit(s) in the instrument and further to processor (52) via the coupling unit. This phenomenon may enable IGS navigation system (50) to determine the location of the distal end of the instrument within a three-dimensional space (i.e., within the head (H) of the patient (P), etc.). To accomplish this, processor (52) executes an algorithm to calculate location coordinates of the distal end of the instrument from the position related signals of the coil(s) in the instrument. Thus, a navigation sensor may serve as a position sensor by generating signals indicating the real-time position of the sensor within three-dimensional space.

Processor (52) uses software stored in a memory of processor (52) to calibrate and operate IGS navigation system (50). Such operation includes driving field generators (64), processing data from the instrument, processing data from operating controls (54), and driving display screen (56). In some implementations, operation may also include monitoring and enforcement of one or more safety features or functions of IGS navigation system (50). Processor (52) is further operable to provide video in real time via display screen (56), showing the position of the distal end of the instrument in relation to a video camera image of the patient's head (H), a CT scan image of the patient's head (H), and/or a computer-generated three-dimensional model of the anatomy within and adjacent to the patient's nasal cavity. Display screen (56) may display such images simultaneously and/or superimposed on each other during the surgical procedure. Such displayed images may also include graphical representations of instruments that are inserted in the patient's head (H), such that the operator may view the virtual rendering of the instrument at its actual location in real time. By way of example only, display screen (56) may provide images in accordance with at least some of the teachings of U.S. Pat. No. 10,463,242, entitled “Guidewire Navigation for Sinuplasty,” issued Nov. 5, 2019, the disclosure of which is incorporated by reference herein, in its entirety. In the event that the operator is also using an endoscope, the endoscopic image may also be provided on display screen (56). The images provided through display screen (56) may help guide the operator in maneuvering and otherwise manipulating instruments within the patient's head (H).

### II. EXAMPLES OF PATIENT TRACKING ASSEMBLIES

In the present example, field generators (64) are in fixed positions relative to the head (H) of the patient (P), such that the frame of reference for IGS navigation system (50) (i.e., the electromagnetic field generated by field generators (64)) does not move with the head (H) of the patient (P). In some instances, the head (H) of the patient (P) may not remain completely stationary relative to field generators (64) throughout the duration of a medical procedure, such that it may be desirable to track movement of the head (H) of the patient (P) during a medical procedure. Examples of patient tracking assemblies that may be used with IGS navigation system (50) are described in greater detail below.

The patient tracking assemblies of the following examples are positioned in the mouth of the patient (P). It should be understood that the positioning of the below described patient tracking assemblies in the mouth of the patient (P) will not necessarily cause an airway obstruction; and may further accommodate intubation of the patient (P) if needed. It should also be understood that, by being positioned in the mouth of the patient (P), the below described patient tracking assemblies may be less susceptible to tracking errors that might otherwise be found in patient tracking assemblies that are mounted externally to the skin of the patient (e.g., inadvertent movement of the patient tracking assembly due to the elasticity of the skin). Moreover, by being positioned in the mouth of the patient (P), the below described patient tracking assemblies may be closer to the skull base and car canals, which may in turn minimize angular/location error that might otherwise be found in patient tracking assemblies that are mounted elsewhere (e.g., on the forehead of the patient). This may be particularly beneficial for procedures where IGS system (50) will be used to track the position of a medical instrument that is inserted into a posterior region of the head (H) of the patient (P) or otherwise near the skull base.

A. Example of Patient Tracking Assembly with Deformable Material

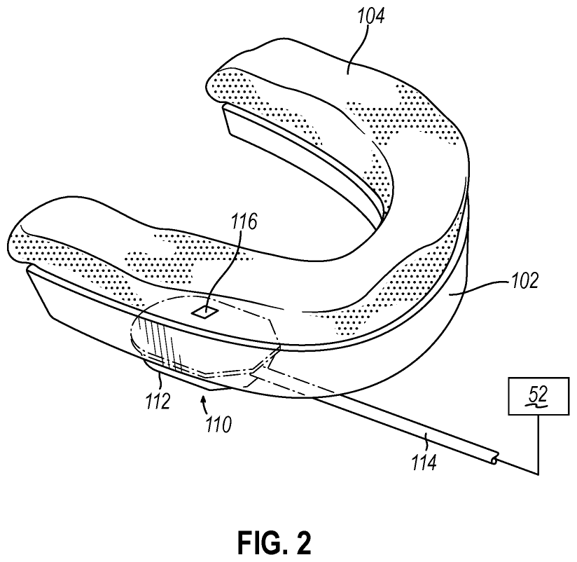

FIGS. 2-5 show an example of a patient tracking assembly (100) that includes a tray (102) containing a deformable material (104) and a sensor assembly (110). In the present example, deformable material (104) is positioned on one side of tray (102) while sensor assembly (110) is positioned on the other side of tray (102). In some other variations, sensor assembly (110) is positioned on the same side of tray (102) as deformable material (104). Tray (102) is sized and configured to generally correspond to an alveolar ridge in the mouth of the patient (P). In some cases, tray (102) is particularly configured to correspond to a maxillary alveolar ridge. In some other cases, tray (102) is particularly configured to correspond to a mandibular alveolar ridge. In still other cases, tray (102) is configured such that tray (102) will correspond to both the maxillary alveolar ridge and the mandibular alveolar ridge, such that the operator may choose the alveolar ridge to which tray (102) should be mounted as will be described in greater detail below.

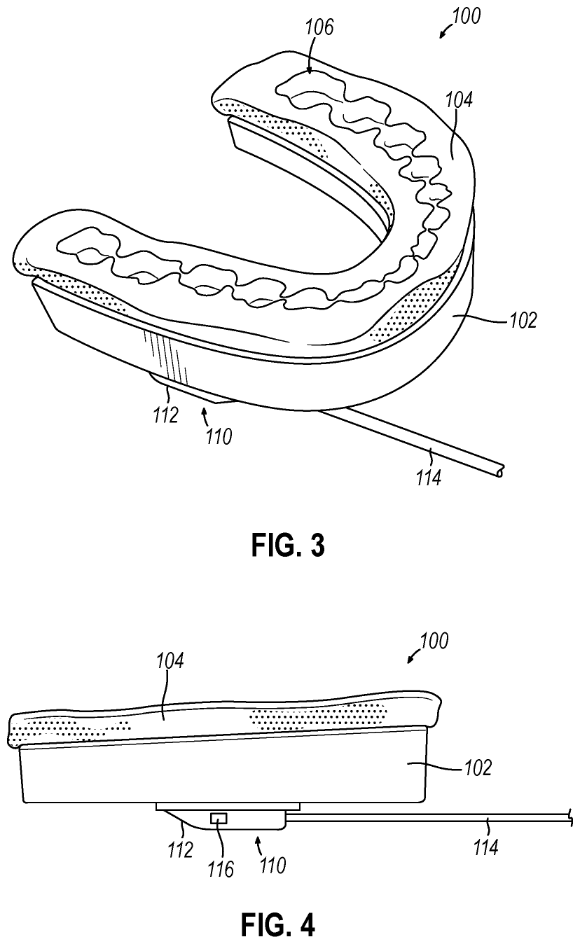

Deformable material (104) may comprise any suitable material or combination of material that may safely receive teeth of the patient. By way of example only, deformable material (104) may comprise a foam, a putty, a wax, an elastomeric material, and/or any other suitable kind(s) of material(s). As shown in FIG. 3, deformable material (104) is configured to deform when the patient (P) bites down on patient tracking assembly (100), such that the teeth of the patient (P) form an impression (106) in deformable material (104). In some versions, deformable material (104) is elastically deformable, such that impression (106) may be formed only temporarily. In some other versions, deformable material (104) is plastically deformable, such that impression (106) is substantially permanent. By way of example only, deformable material (104) may be initially deformable; and may then harden after curing for a certain period. Regardless of the composition of deformable material (104), tray (102) and deformable material (104) of the present example are configured such that patient tracking assembly (100) may accommodate patients of various sizes and having various dental configurations. In other words, patient tracking assembly (100) of the present example may be considered as a universal device, such that, at least until impression (106) is made in deformable material (104), patient tracking assembly (100) is not specifically configured for a particular patient.

Sensor assembly (110) of the present example includes a body (112) and a cable (114). A position sensor (116) is housed within body (112). Position sensor (116) is configured to generate signals indicating the real-time position of position sensor (116) in response to an alternating electromagnetic field generated by field generators (64). By way of example only, position sensor (116) may comprise one or more coils. The signals generated by position sensor (116) are communicated along cable (114) to processor (52), such that processor (52) may process signals from position sensor (116) to determine the real-time position of position sensor (116) in three-dimensional space. In some other versions, patient tracking assembly (100) is configured to communicate the signals from position sensor (116) wirelessly, such that cable (114) may be omitted in some versions.

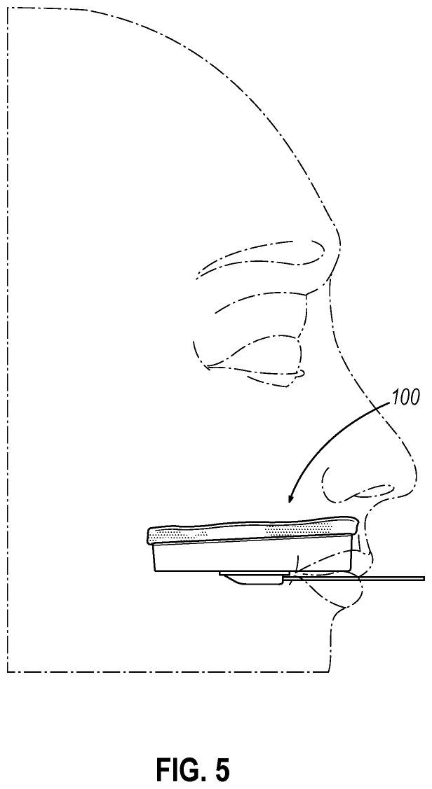

As shown in FIG. 5, patient tracking assembly (100) may be inserted into the mouth of the patient (P) and positioned over an alveolar ridge of the patient (P). In the example shown in FIG. 5, patient tracking assembly (100) is positioned over the maxillary alveolar ridge; though in other scenarios patient tracking assembly (100) may be positioned over the mandibular alveolar ridge. In some cases, the operator presses patient tracking assembly (100) onto the mandibular alveolar ridge, thereby pressing the dentition of the patient (P) into deformable material (104). In addition, or in the alternative, the patient (P) may be instructed to bite down onto patient tracking assembly (100) to thereby press the dentition of the patient (P) into deformable material (104). In either scenario, with the dentition of the patient (P) into deformable material (104), patient tracking assembly (100) may remain substantially firmly fixed in the head (H) of the patient (P). With such substantial fixation in the head (H) of the patient (P), patient tracking assembly (100) may move unitarily with the head (H) of the patient (P). Accordingly, signals from position sensor (116) may effectively indicate the real-time position of the head (H) of the patient in three-dimensional space.

After patient tracking assembly (100) is installed in the mouth of the patient (P), an operator may insert one or more position sensor equipped medical instruments (e.g., ENT shaver, suction cannula, balloon dilation catheter, electrosurgical instrument, etc.) into the head (H) of the patient (P). Signals from position sensors of such instruments may be communicated to processor (52), thereby enabling processor (52) to determine the real-time positions of such instruments in three-dimensional space. With processor (52) knowing the real-time position of the head (H) of the patient (P) in three-dimensional space based on signals from position sensor (116), and with processor (52) knowing the real-time position of a medical instrument in three-dimensional space based on signals from one or more position sensors in the medical instrument, processor (52) may accurately determine the real-time position of the medical instrument in the head (H) of the patient (P). Processor (52) may thereby drive display screen (56) to display an indicator (e.g., crosshairs, etc.) showing the real-time position of the medical instrument in the head (H) of the patient (P). By way of example only, processor (52) may drive display screen (56) to display an indicator (e.g., crosshairs, etc.) to show the real-time position of the medical instrument in the head (H) of the patient (P) as an overlay on one or more preoperative images (e.g., CT scans, etc.) of at least a portion of the head (H) of the patient (P). By way of further example only, processor (52) may drive display screen (56) to display an indicator (e.g., crosshairs, etc.) to show the real-time position of the medical instrument in the head (H) of the patient (P) as an overlay on a 3D digital model of at least a portion of the head (H) of the patient (P). Alternatively, processor (52) may drive display screen (56) to indicate the real-time position of the medical instrument in the head (H) of the patient (P) in any other suitable fashion.

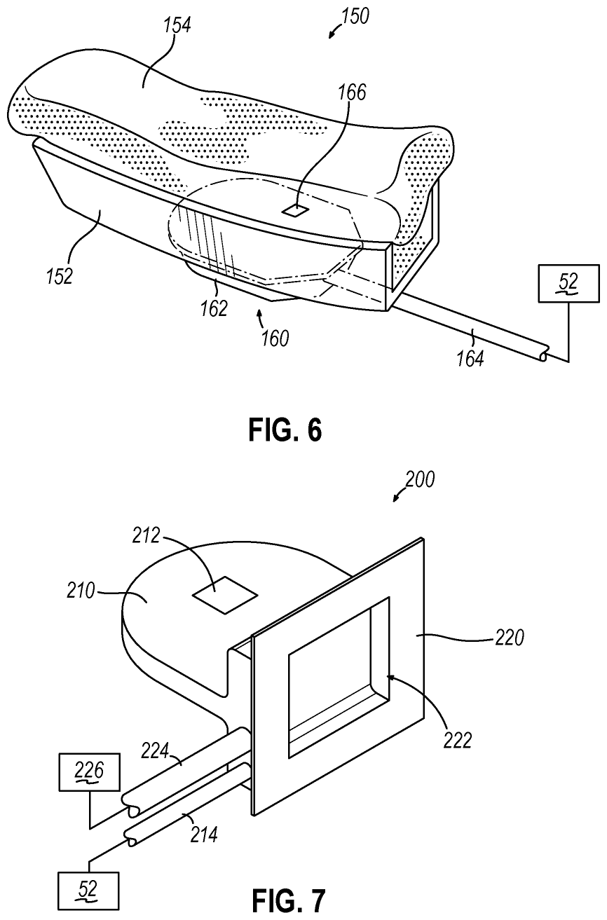

FIG. 6 shows another example of a patient tracking assembly (150). Patient tracking assembly (150) of this example includes a tray (152) containing a deformable material (154) and a sensor assembly (160). Sensor assembly (160) includes a body (162), a cable (164), and a position sensor (166). Sensor assembly (160) is thus configured and operable just like sensor assembly (110) described above. Deformable material (154) of patient tracking assembly (150) is also configured and operable like deformable material (104) of patient tracking assembly (100). Tray (152) of patient tracking assembly (150) is similar to tray (102) of patient tracking assembly (100), except that tray (152) of this example is configured to extend along only a portion of an alveolar ridge of a patient (P). By contrast, tray (102) of patient tracking assembly (100) defines an arch configured to complement a full arch of an alveolar ridge of the patient.

In some cases, tray (152) is particularly configured to correspond to an anterior region of an alveolar ridge. In some other cases, tray (152) is particularly configured to correspond to a posterior region of an alveolar ridge. In still other cases tray (152) is configured such that tray (152) will correspond to both the anterior region of an alveolar ridge and the posterior region of an alveolar ridge, such that the operator may choose the region of the alveolar ridge to which tray (152) should be mounted. It should also be understood that tray (152) may correspond to a region of a mandibular alveolar ridge, a region of a maxillary alveolar ridge, or both.

B. Example of Patient Tracking Assembly with Bitewing and Suction

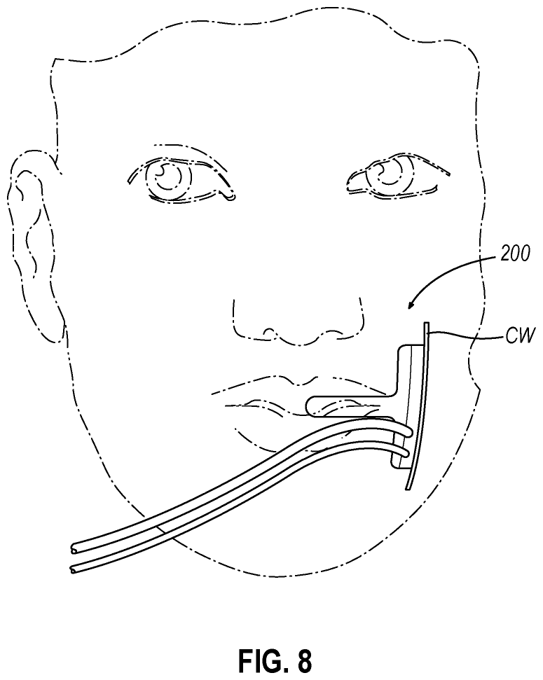

While patient tracking assemblies (100, 150) described above are configured to be substantially fixed within a mouth of a patient (P) through engagement between the dentition of the patient (P) and deformable material (104, 154), it may be desirable to provide some other form of fixation between a patient tracking assembly and a mouth of the patient (P). To that end, FIGS. 7-8 show another example of a patient tracking assembly (200) that is fixed within the mouth of the patient (P) through biting and/or suction. Patient tracking assembly (200) of this example includes a bitewing (210) and a suction base (220). In the present example, bitewing (210) and suction base (220) are substantially perpendicular to each other. Alternatively, bitewing (210) and suction base (220) may define any other suitable angle(s) relative to each other.

Bitewing (210) is configured to be positioned between the maxillary and mandibular jaws of the patient (P), such that the patient (P) may bite down on bitewing (210) as described in greater detail below. In some versions, a biting wax element or other deformable material is secured to bitewing (210) to further promote firm engagement between the teeth of the patient and bitewing (210) when the patient bites down on bitewing (210).

A position sensor (212) is contained within bitewing (210). Position sensor (212) is configured to generate signals indicating the real-time position of position sensor (212) in response to an alternating electromagnetic field generated by field generators (64). By way of example only, position sensor (212) may comprise one or more coils. The signals generated by position sensor (212) are communicated along cable (224) to processor (52), such that processor (52) may process signals from position sensor (212) to determine the real-time position of position sensor (212) in three-dimensional space. In some other versions, patient tracking assembly (200) is configured to communicate the signals from position sensor (212) wirelessly, such that cable (214) may be omitted in some versions.

Suction base (220) of the present example defines a laterally presented cavity (222). Cavity (222) is in fluid communication with a conduit (224) (e.g., a flexible tube), which is in further fluid communication with a suction source (226). By way of example only, suction source (226) may comprise a pump, a syringe, or any other suitable structure that is operable to communicate suction to conduit (224). Conduit (224) is configured to communicate such suction to cavity (222).

In operation, patient tracking assembly (200) may be positioned in the mouth of the patient (P) as shown in FIG. 8. In particular, bitewing (210) may be positioned between the jaws of the patient (P), with suction base (220) being positioned against the cheek wall (CW) of the patient (P). In some scenarios, patient tracking assembly (200) is positioned at the third molar region of the patient (P). The patient (P) is instructed to bite down on bitewing (210); and suction is applied to cavity (222) by suction source (226) and conduit (224). This suction may be further applied against the cheek wall (CW), such that suction base (220) is substantially fixed to the cheek wall (CW) due to the suction. In some versions, at least a portion of suction base (220) (e.g., a gasket or a rim) is deformable to further promote a fluid-tight seal between suction base (220) and the cheek wall (CW).

It should be understood that the combination of suction applied to the cheek wall (CW) by suction base (220) and the patient (P) biting on bitewing (210) may substantially fix the position of patient tracking assembly (200) in the head (H) of the patient (P). In some scenarios, patient tracking assembly (200) may be substantially fixed in the head (H) of the patient (P) by the patient (P) biting on bitewing (210) alone, such that suction need not necessarily be applied to the cheek wall (CW) by suction base (220) in all scenarios. Moreover, suction base (220) may be omitted in some variations. It should also be understood that patient tracking assembly (200) may be substantially fixed in the head (H) of the patient (P) by suction applied to the cheek wall (CW) by suction base (220) alone, such that the patient need not necessarily bite down on bitewing (210) in all scenarios. Moreover, bitewing (210) may be omitted in some variations. In such variations, position sensor (212) may be integrated into suction base (220). It should also be understood that some other variations that include bitewing (210) may further provide position sensor (212) in suction base (220) instead of bitewing (210).

In the foregoing examples, a patient tracking assembly (100, 150, 200) is secured relative to the patient (P) via deformable material (104, 154), via a bitewing (210), and/or via a suction base (220). As yet another illustrative example, a patient tracking assembly may be secured relative to the patient (P) via a tooth band, such as a molar band that is used in orthodontic procedures. Such a tooth band may be wrapped around one or more teeth and thus be secured to the one or more teeth. In such versions, a position sensor like position sensors (116, 166, 212) may be integrated with or secured to the tooth band.

### III. EXAMPLE OF REGISTRATION WITH PATIENT TRACKING ASSEMBLY

One function that may be performed by IGS system (50) is obtaining one or more reference points that may be used to correlate various preoperatively obtained images with a patient's actual position during a procedure. This act may be referred to as patient registration.

Such registration may be performed by using a positionally tracked instrument (e.g., a registration probe whose tip position may be detected in three-dimensional space) to trace or touch one or more positions on a patient's face. At each touch point, IGS system (50) may register that point in three-dimensional space; and, using a number of registered points, determine the position of the affected area in three-dimensional space. Once the affected area is fully mapped or registered, it may be correlated with preoperative images in order to provide a seamless IGS experience across varying types of preoperative images during the performance of the procedure.

In some cases, an operator may press a registration probe against the skin of the patient (P) with too much force, which may cause the skin to dent or deform. This may compromise the accuracy of any registration points captured at sites where the skin was dented or deformed. It may therefore be desirable to provide a form of registration that avoids the risk of inaccuracies caused by skin deformation. To that end, in lieu of using a registration probe or other device to achieve registration of the patient (P) with one or more preoperative images, registration may be carried out using a patient tracking assembly (100, 200). For instance, one or more of the preoperative images (e.g., CT scans, etc.) may be captured while patient tracking assembly (100, 200) is already installed in the mouth of the patient (P). Patient tracking assembly (100, 200) may include one or more fiducial points that may be readily identified in the preoperative images, such that processor (52) may effectively register the patient (P) with the preoperative images based on detected fiducial points of patient tracking assembly (100, 200).

### IV. EXAMPLES OF COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) a tray, the tray being configured to correspond to at least a portion of an alveolar ridge of a patient; (b) a deformable material, the deformable material being configured to receive dentition of the alveolar ridge of the patient and thereby secure the tray relative to the alveolar ridge of the patient; and (c) a position sensor fixed relative to the tray, the position sensor being configured to generate a signal indicating a real-time position of the position sensor within three-dimensional space.

### Example 2

The apparatus of Example 1, the tray defining an arch configured to complement a full arch of an alveolar ridge of the patient.

### Example 3

The apparatus of any of Examples 1 through 2, the deformable material comprising a material selected from the group consisting of a foam, a putty, a wax, and an elastomeric material.

### Example 4

The apparatus of any of Examples 1 through 3, the deformable material being elastically deformable.

### Example 5

The apparatus of any of Examples 1 through 3, the deformable material being plastically deformable.

### Example 6

The apparatus of any of Examples 1 through 5, the position sensor comprising one or more coils.

### Example 7

The apparatus of Example 6, the one or more coils being configured to generate a signal indicating a real-time position of the position sensor within three-dimensional space in response to an electromagnetic field.

### Example 8

The apparatus of any of Examples 1 through 7, further comprising a cable, the cable being able to couple the position sensor with a processor.

### Example 9

The apparatus of any of Examples 1 through 8, the tray including a first side and a second side, the deformable material being positioned on the first side of the tray, the position sensor being positioned on the second side of the tray.

### Example 10

An apparatus, comprising: (a) a body, the body defining a cavity, the body being configured to fit in a mouth of a patient, adjacent to a cheek wall in the mouth of the patient; (b) a position sensor secured relative to the body, the position sensor being configured to generate a signal indicating a real-time position of the position sensor within three-dimensional space; and (c) a suction conduit in fluid communication with the cavity, the suction conduit being configured to apply suction to the cavity to thereby secure the body to the cheek wall in the mouth of the patient.

### Example 11

The apparatus of Example 10, the body further comprising a bitewing, the bitewing being configured to fit between teeth of opposing jaws of the patient.

### Example 12

The apparatus of Example 11, the bitewing including a deformable material.

### Example 13

The apparatus of Example 12, the deformable material comprising a wax material.

### Example 14

The apparatus of any of Examples 11 through 12, the body further comprising a suction base, the cavity being defined in the suction base.

### Example 15

The apparatus of Example 14, the bitewing being substantially perpendicular to the suction base.

### Example 16

The apparatus of any of Examples 14 through 15, at least a portion of the suction base being deformable to at least partially conform to the cheek wall.

### Example 17

The apparatus of any of Examples 11 through 16, the position sensor being positioned in the bitewing.

### Example 18

The apparatus of any of Examples 10 through 17, the position sensor comprising one or more coils.

### Example 19

The apparatus of Example 18, the one or more coils being configured to generate a signal indicating a real-time position of the position sensor within three-dimensional space in response to an electromagnetic field.

### Example 20

The apparatus of any of Examples 10 through 19, further comprising a cable, the cable being able to couple the position sensor with a processor.

### Example 21

A method comprising: (a) inserting an apparatus into a mouth of a patient, the apparatus comprising: (i) a tray, the tray being configured to correspond to at least a portion of an alveolar ridge of a patient, (ii) a deformable material, the deformable material being configured to receive dentition of the alveolar ridge of the patient and thereby secure the tray relative to the alveolar ridge of the patient, and (iii) a position sensor fixed relative to the tray, the position sensor being configured to generate a signal indicating a real-time position of the position sensor within three-dimensional space; and (b) securing the apparatus to the patient, the apparatus being secured to the patient through engagement between dentition of the patient and the deformable material.

### Example 22

The method of Example 21, the act of securing the apparatus to the patient comprising pressing the deformable material against the dentition of the patient.

### Example 23

The method of any of Examples 21 through 22, the act of securing the apparatus to the patient comprising instructing the patient to bite down on the deformable material.

### Example 24

The method of any of Examples 21 through 23, further comprising operating an image guided surgery system, the image guided surgery system tracking a real-time position of the head of the patient based on signals from the position sensor during the act of operating the image guided surgery system.

### Example 25

The method of Example 24, the act of operating an image guided surgery system further comprising inserting a medical instrument into the head of the patient, the medical instrument including a position sensor, the image guided surgery system tracking a real-time position of the medical instrument in the head of the patient based on signals from the position sensor of the medical instrument while the medical instrument is inserted into the head of the patient.

### Example 26

A method, comprising: (a) inserting an apparatus into a mouth of a patient, the apparatus comprising: (i) a body, the body defining a cavity, (ii) a position sensor secured relative to the body, the position sensor being configured to generate a signal indicating a real-time position of the position sensor within three-dimensional space, and (iii) a suction conduit in fluid communication with the cavity; and (b) securing the apparatus to the patient, the act of securing the apparatus to the patient comprising: (i) positioning the cavity adjacent to a cheek wall in the mouth of the patient, and (ii) applying suction to the cavity via the suction conduit, thereby securing the body to the cheek wall.

### Example 27

The method of Example 26, the body further comprising a bitewing, the method further comprising positioning the bitewing between teeth of opposing jaws of the patient.

### Example 28

The method of Example 27, the act of securing the apparatus to the patient further comprising instructing the patient to bite down on the bitewing.

### Example 29

The method of any of Examples 26 through 28, further comprising operating an image guided surgery system, the image guided surgery system tracking a real-time position of the head of the patient based on signals from the position sensor during the act of operating the image guided surgery system.

### Example 30

The method of Example 29, the act of operating an image guided surgery system further comprising inserting a medical instrument into the head of the patient, the medical instrument including a position sensor, the image guided surgery system tracking a real-time position of the medical instrument in the head of the patient based on signals from the position sensor of the medical instrument while the medical instrument is inserted into the head of the patient.

### V. MISCELLANEOUS

It should be understood that any of the teachings, expressions, embodiments, examples, etc. described herein may be combined with any of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus, comprising:
(a) a tray, the tray being configured to correspond to at least a portion of an alveolar ridge of a patient;
(b) a deformable material, the deformable material being configured to receive dentition of the alveolar ridge of the patient and thereby secure the tray relative to the alveolar ridge of the patient; and
(c) a position sensor fixed relative to the tray, the position sensor being configured to generate a signal indicating a real-time position of the position sensor within three-dimensional space.

2. The apparatus of claim 1, the tray defining an arch configured to complement a full arch of an alveolar ridge of the patient.

3. The apparatus of claim 1, the deformable material comprising a material selected from the group consisting of a foam, a putty, a wax, and an elastomeric material.

4. The apparatus of claim 1, the deformable material being elastically deformable.

5. The apparatus of claim 1, the deformable material being plastically deformable.

6. The apparatus of claim 1, the position sensor comprising one or more coils.

7. The apparatus of claim 6, the one or more coils being configured to generate a signal indicating a real-time position of the position sensor within three-dimensional space in response to an electromagnetic field.

8. The apparatus of claim 1, further comprising a cable, the cable being able to couple the position sensor with a processor.

9. The apparatus of claim 1, the tray including a first side and a second side, the deformable material being positioned on the first side of the tray, the position sensor being positioned on the second side of the tray.

10. An apparatus, comprising:
(a) a body, the body defining a cavity, the body being configured to fit in a mouth of a patient, adjacent to a cheek wall in the mouth of the patient;
(b) a position sensor secured relative to the body, the position sensor being configured to generate a signal indicating a real-time position of the position sensor within three-dimensional space; and
(c) a suction conduit in fluid communication with the cavity, the suction conduit being configured to apply suction to the cavity to thereby secure the body to the cheek wall in the mouth of the patient.

11. The apparatus of claim 10, the body further comprising a bitewing, the bitewing being configured to fit between teeth of opposing jaws of the patient.

12. The apparatus of claim 11, the bitewing including a deformable material.

13. The apparatus of claim 12, the deformable material comprising a wax material.

14. The apparatus of claim 11, the body further comprising a suction base, the cavity being defined in the suction base.

15. The apparatus of claim 14, at least a portion of the suction base being deformable to at least partially conform to the cheek wall.

16. The apparatus of claim 11, the position sensor being positioned in the bitewing.

17. The apparatus of claim 10, the position sensor comprising one or more coils

18. The apparatus of claim 10, further comprising a cable, the cable being able to couple the position sensor with a processor.

19. A method comprising:
(a) inserting an apparatus into a mouth of a patient, the apparatus comprising:
(i) a tray, the tray being configured to correspond to at least a portion of an alveolar ridge of a patient,
(ii) a deformable material, the deformable material being configured to receive dentition of the alveolar ridge of the patient and thereby secure the tray relative to the alveolar ridge of the patient, and
(iii) a position sensor fixed relative to the tray, the position sensor being configured to generate a signal indicating a real-time position of the position sensor within three-dimensional space; and
(b) securing the apparatus to the patient, the apparatus being secured to the patient through engagement between dentition of the patient and the deformable material.

20. A method, comprising:
(a) inserting an apparatus into a mouth of a patient, the apparatus comprising:
(i) a body, the body defining a cavity,
(ii) a position sensor secured relative to the body, the position sensor being configured to generate a signal indicating a real-time position of the position sensor within three-dimensional space, and
(iii) a suction conduit in fluid communication with the cavity; and
(b) securing the apparatus to the patient, the act of securing the apparatus to the patient comprising:
(i) positioning the cavity adjacent to a cheek wall in the mouth of the patient, and
(ii) applying suction to the cavity via the suction conduit, thereby securing the body to the check wall.
